(19) United States
(12) Patent Application Publication
(10) Pub. No.: US 2011/0221598 A1
(43) Pub. Date: Sep. 15, 2011

(54) device for avoiding sudden infant death

(76) Inventors: Johannes Eschler, Ditzingen (DE); Michael Dambier, Bretten (DE)

(21) Appl. No.: 13/063,170

(22) PCT Filed: Jul. 30, 2009
(86) PCT No.: PCT/EP09/59903

§ 371 Date: May 16, 2011

(30) Foreign Application Priority Data
Sep. 12, 2008 (DE) 102008042017.4

(51) Int. Cl. G08B 23/00; A61B 5/00

(52) U.S. Cl. 340/575; 600/300

(57) ABSTRACT

A device for avoiding sudden infant death. It includes a recording device for recording at least one infant-specific state parameter and an awakening device for waking up the infant as a function of a value of the recorded state parameter.

### FIELD OF THE INVENTION

The present invention relates to a device for avoiding sudden infant death.

### BACKGROUND INFORMATION

In spite of the knowledge of various risk factors, In the final analysis the causes of sudden infant death (SID) have still not been completely cleared up. According to Federal German reporting, in an international comparison, Germany forms one of the tail lights in SID avoidance. This circumstance is to be countered by using an improved information strategy, and the sensitization of the population to this problem. In this instance, based on numerous studies, the following recommendations, among others, are being given to parents:

a) Avoiding the Prone Position of the Sleeping Infant

This recommendation was given since numerous SID victims were laid to sleep in the prone position or the lateral position and were found dead in the prone position. It was derived from studies that the risk rises greatly if the infant is laid to sleep in the prone position. But even if the infant is, in fact, laid to sleep in the supine position or the lateral position, the danger still exists that the infant will turn into the prone position while sleeping, so that, even assuming correct bedding of the infant, a reduced but still a significant residual risk for SID still remains. Physiopathological investigations have shown that, because of the prone position, frequently a lesser brain stem circulation takes place. For this reason, infants in the prone position apparently breathe in a more shallow manner, and the cough reflex is marked more weakly. In the case of the prone position, investigations of the bodies of the cervical vertebra demonstrated the occurrence of an increased angle between the first and the second body of the cervical vertebra. It is suspected that this, too, is a factor favoring SID.

b) Protection from Overheating the Infant

It was proved in studies that hyperthermia of the infant has the effect of a 3.5 times greater risk for SID. As a control possibility, it is recommended that the infant should be warm in the nape of the neck and between the shoulder blades, it is true, but should in no case be sweaty.

c) The Use of a Sleeping Bag

SID victims have often been found covered by a blanket or a pillow. In this context, the neck and the mouth were found pressed against the cover, the pillow or a large, cuddly toy, which leads to the conclusion that the SID was caused by rebreathing or asphyxia. When infants sleep in the bed of the parents, there is the risk, added to the abovementioned ones, of the infant's being covered by the body of the father or the mother. The recommendation is to use a special sleeping bag whose neck opening is not greater than the circumference of the head of the infant. Such a sleeping bag is also suitable for avoiding, to the greatest extent possible, the turning over of an infant bedded in the supine position into the prone position that is connoted with risk (see recommendation a)).

d) Renunciation of Smoking

Approximately 30% of SID cases were associated in studies with tobacco smoke exposure during and after pregnancy.

At this time, various products are available on the market that are intended to be used for avoiding SID. Among these are particularly sleeping bags for infants, thermometers for determining and evaluating the skin temperature of the infant, as well as movement monitors based on a pressure-sensitive mat, which sound an alarm if the infant does not move for more than 20 seconds. Breathing monitors are also available for monitoring the breathing based on a sensor mat, which sound an alarm when breathing is interrupted for more than 20 seconds or when the infant inhales fewer than 10 times per minute. Finally, there are devices (at this time only in the clinical field), by which a plurality of vital signs of an infant are able to be monitored. Besides the apparatus complexity and cost intensiveness of such medicinal technology equipment (such as devices for cardiorespiratory monitoring), in this instance, too, certain prevention of SID has not been proven, so that the application of such devices in the private sector does not appear to be recommended.

### SUMMARY OF THE INVENTION

A basic aspect of the exemplary embodiments and/or exemplary methods of the present invention is to wake up the infant for safety's sake, if risk factors favoring SID are present. Therefore, a device is provided for avoiding sudden infant death which includes recording device for recording at least one infant-specific state parameter and an awakening device for awakening the infant as a function of the value of the recorded state parameter.

Such an awakening device provides an automated awakening function which ensures a safe and, at the same time, cost-effective avoidance of SID, independently of human monitoring or intervention (such as testing the vital signs of the infant and awakening by the parents, if necessary. The exemplary embodiments and/or exemplary methods of the present invention thereby attacks the problem of SID at its roots, namely the sleep of the infant itself. For, an awake infant is not endangered by SID, at least according to current knowledge. It is assumed that the state of sleeping of the infant is the indispensable prerequisite for the appearance of SID. The reason is that, exclusively in the sleeping state, the vegetative nervous system has the upper hand, and, in the subconscious, controls only the most important vital functions, such as breathing, heart beat, etc., so that outer influence factors, which are able to endanger these primary vital functions, such as heat, lack of air, etc., cannot be perceived by the conscious, and thus no deliberate countermeasures, such as thrashing about until free, turning over, crying out, etc., can be initiated. Since, however, particularly in the case of infants, sufficient sleeping time is indispensable for the body, it is not expedient regularly to wake up the infant, such as at predetermined time periods. At this point, another advantage of the exemplary embodiments and/or exemplary methods of the present invention becomes clear, for the awakening device is equipped to wake up the infant as a function of a value of an infant-specific state parameter recorded by a recording device, whereby the awakening process is supported by an infant-related decision basis, which advantageously reduces the wake-up frequency.

The device according to the present invention may also have a checking device to check whether the value of the recorded state parameter is inside or outside a predefined value range, which includes all the values of the state parameter that represent a risk for the occurrence of sudden infant death, the awakening device being equipped to wake up the infant if at least this one value of the recorded state parameter lies within this predefined value range. It is particularly advantageous if the awakening device is equipped to wake up the infant only if at least this value of the recorded state parameter lies within this predefined value range. The waking up is able to take place immediately after the presence of the risk value, in this instance, or at a constant or variable time delay after the presence of the risk value. It is also possible to make the awakening process a function of the simultaneous occurrence of a plurality of specifiable risk values for a plurality of different state parameters.

The advantage of this specific embodiment is that the frequency of the awakening is able to be limited to an essential measure, and consequently, the sleep duration is able to be optimized, for the infant is only awakened if there exists a risk of the occurrence of SID.

According to one additional advantageous embodiment of the present invention, the device also includes a warning device for what may be the immediate outputting of a warning if it has been determined by the checking device that the value of the state parameter recorded lies within the specified value range, the awakening device being equipped to wake up the infant only after the expiration of a predetermined time period after the output of the warning.

The advantage of this specific embodiment is that, because of the warning, first of all caregivers of the infant, such as parents, can be alerted and notified that the checking device has determined an indication for a potentially existing SID risk. The output of this warning may take place in such a way that only the caregiver is alerted and awakened if necessary, without the sleeping infant being able to perceive the warning. This may be implemented by the warning device being located spatially separate from the sleeping infant (e.g. in the bedroom of the parents), and there drawing attention of the parents or waking them up using an acoustical and/or visual warning signal (e.g. a loud tone and/or a bright light). The warning instruction, in this context, may be transmitted either wire-bound or wireless (e.g. infrared-based or radio wave-based) from the checking device or from a central control device, connected to it, to the warning device. If infant and parents were to sleep close together (e.g. in one bed), one could use a vibrating warning signal, which does, in fact, vibrate the parents awake, but does, at first, not bother the infant. Because of the time delay of the awakening process, enough time remains for the parents to obtain personally an idea of the state of health of the sleeping infant, and perhaps to deactivate the awakening device before the expiration of the delay time, if the parents determine that everything is in order. For, since the selection of the risk value range for the safety of the infant should rather be too great than too little, time and again there will be a “blind alarm”, i.e. the infant would be awakened although there is no acute danger. This cannot be avoided, based on the complexity that was explained in the introductory part of the description, and the contexts of the SID problem that have not yet been fully cleared up.

In this way, compared to the previously described specific embodiment, the wake-up frequency of the infant may be reduced even further to the absolutely essential measure, and consequently, the sleeping duration of the infant is maximized.

In this specific embodiment, it is also possible to make the duration of the time delay of the awakening process dependent upon which and/or how many infant-specific state parameters have a value in the established risk value range, and/or at how far a distance such a risk value is from the just still admissible value of the respective state parameter. The more weight the respective state parameter carries for the SID risk, the more state parameters have a value in the established risk value range and the farther removed such a risk value is from the just still admissible value of the respective state parameter, the shorter should be the duration of the time delay of the awakening process. Thereby, one may react in an appropriate time in each case, depending on the seriousness of the SID risk.

The time delay of the awakening process may also be utilized in a still different manner. For, if the infant-specific state parameter is normalized within the time delay duration from a value within the risk value range to a value outside the risk value range, the awakening device is able to be deactivated by itself, i.e. automatically, without human intervention, so that the infant is not awakened unnecessarily.

According to one specific embodiment of the present invention, the device in order to prevent sudden infant death also includes an acknowledgment device for acknowledging taking note of the warning output by the warning device, the awakening device being able to be deactivated by acknowledging taking note of the warning within the predetermined time period, so that the infant is not awakened by the acknowledgment of the warning made within the predetermined time period.

This specific embodiment combines the advantage of the certainty that the infant potentially endangered by SID is awakened in case the parents do not react to the warning, with the advantage of the maximized sleeping duration of the infant (see above).

By contrast to the positioning of the warning device, in the case of the acknowledgment device it is of advantage if it is located in the vicinity of the sleeping infant. This ensures that the parents actually have to make their way to the infant, in order to break off the automatic nature of the awakening process. Consequently, it is ensured that the parents do not break off the automated awakening process without seeing to the infant.

For the practical embodiment of the awakening device in the specific embodiments described above, one might consider, for example, a vibrating mat that is laid underneath the infant. This vibrating mat is in a position to generate vibrations and to transfer these to the infant, so that the infant wakes up as a result of these vibrations. However, any other embodiment, that is suitable for certainly awakening the infant, is possible too. Devices are also conceivable in this context, for instance, which are equipped to wake up the infant using one or more sound and/or light emissions. A combination of the methods named increases the probability that the infant is actually awakened in response to the activation of the awakening device.

For the practical embodiment of the recording device and for the selection of the state infant-specific parameters that are to be recorded, many possibilities arise. Thus, for example, all the parameters mentioned in the introductory part of the specification, individually or in any combination, are also suitable within the scope of the exemplary embodiments and/or exemplary methods of the present invention, for the purpose of SID prevention. Among these parameters are, among others, the temperature and/or moisture of at least one body part of the infant (which in this case may be in the nape of the neck and/or between the shoulder blades), respiration (absolute breathing activity and/or breathing frequency) and heart beat frequency. Among the possible parameters are also any temporal change rates of the quantities named.

One further advantageous embodiment of the recording device includes at least one sensor unit for determining the body position of the infant. As was explained in detail at the outset, the body position of the infant, and in this case in particular the distinction between the prone position and the non-prone position, is indeed a criterion having great influence on the occurrence of SID.

According to one special form of a position sensor, one might consider an acceleration sensor. This acceleration sensor may be fastened, for instance, in the area of the infant's chest, on its clothing. Thereafter, the infant is laid to sleep in the supine position, that is at low risk for SID. Now, if the infant turns on its side or its belly while sleeping, this corresponds to a rotational motion about the longitudinal axis of the infant body, and thus about the longitudinal axis of the appropriately oriented acceleration sensor. The acceleration, which this rotational motion effects, is able to be measured by the acceleration sensor. In this case, the radially acting acceleration component with respect to the longitudinal axis is measured, which is very specific for the approximate circular motion of the body about its longitudinal axis. In that way, the rotation into the prone position, which favors SID, is able to be distinguished from other harmless motions of the infant (such as thrashing about, which rather brings about rapidly changing acceleration forces in the axial direction), which decidedly reduces the frequency of a “blind alarm”.

According to a second special form of a position sensor, one might consider a gravity sensor. In this case, too, the gravity sensor may be fastened, for instance, in the area of the infant's chest, on its clothing. Thereafter, the infant is again laid to sleep in the supine position, that is at low risk for SID. Gravity in the position of the supine position of the infant then amounts to, for instance, +1 g on the x axis. If the infant turns on its side, the direction of gravity changes in the sensor (e.g. +1 g or −1 g on the y axis). If at any time the infant then lies on its stomach, the direction of gravity in the sensor is at least approximately opposite to the direction of gravity in the sensor during the initial measurement in the supine position, that is, for instance, −1 g on the x axis. This makes possible an exact association of the position of the infant with the sensor data. Thrashing about or other motions of the infant effect only small changes in the direction of gravity in the sensor.

According to a third special possibility of determining the infant's body position, a heat image camera is proposed for taking a heat image of the infant. For, with the aid of a two-dimensional heat image, one is able to monitor not only the temperature of the infant (see also the above statements on measuring the temperature and/or the moisture of at least one part of the infant's body), but also its position. Heat image data are able to be evaluated via an image processing device, with the aid of suitable algorithms (e.g. outline or shape recognition by comparison to previously stored samples), and thus the position of the infant is able to be determined. In addition, the degree of coverage of the infant may be determined in that, on the heat image, uncovered body locations such as the head, hands, etc., become clearly emphasized. Other body regions covered by clothing, a blanket or a pillow, because of their heat development, also become differentiated from the cooler surroundings, but not as greatly as the uncovered body locations.

Further features and advantages of specific embodiments of the present invention result from the following description with reference to the enclosed figures.

### BRIEF DESCRIPTION OF THE FIGURES

FIG. 1 shows a first specific embodiment of the present invention.

FIG. 2 shows a second specific embodiment of the present invention.

FIG. 3 shows a third specific embodiment of the present invention.

FIG. 4 shows a fourth specific embodiment of the present invention.

### DETAILED DESCRIPTION

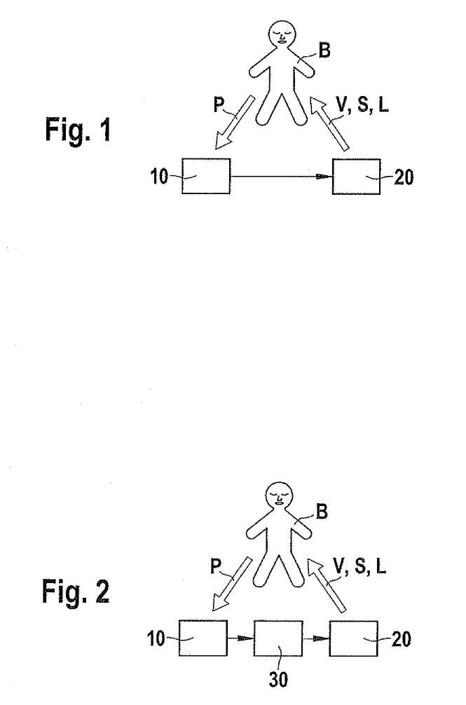

In a schematic manner of illustration, FIG. 1 makes clear an initial form of the exemplary embodiments and/or exemplary methods of the present invention. According to this, a corresponding device for avoiding sudden infant death includes recording device 10 for recording at least one infant-specific state parameter P and an awakening device 20 for awakening the infant B as a function of a value of the recorded state parameter P. The current values of recorded state parameter P reach recording device 10 via sensors (that are not shown here). Waking up the infant may be done by applying vibrations V, sound emissions S and/or light emissions L to infant B. Because the awakening device is equipped to wake up the infant as a function of an infant-specific state parameter recorded by a recording device, the awakening process is supported by an infant-related decision base, which reduces the awakening frequency in an advantageous manner.

FIG. 2 shows an embodiment of the device according to the present invention that builds up on the specific embodiment of FIG. 1. The device also has a checking device 30 to check whether the value of the recorded state parameter P is inside or outside a predefined value range, which includes all the values of state parameter P that represent a risk for the occurrence of sudden infant death. Awakening device 20 is equipped to wake up infant B if at least this one value of the recorded state parameter P lies within this predefined value range. The advantage of this specific embodiment is that the frequency of awakening is able to be limited to an essential measure, and consequently, the sleep duration is able to be optimized, for the infant is only awakened if there exists a risk of the occurrence of SID.

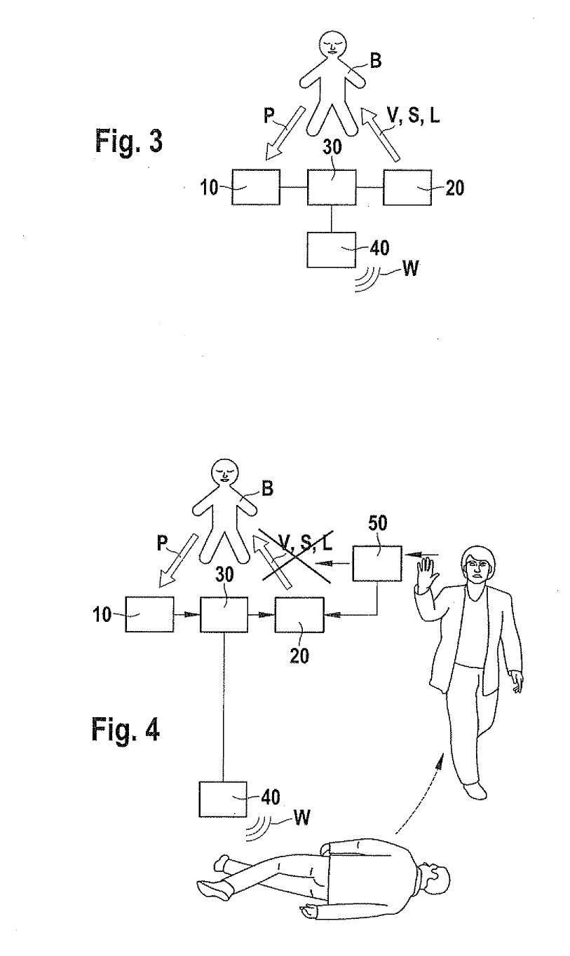

FIG. 3 shows an additional embodiment of the device of the present invention that builds up on the two previously described specific embodiments. Accordingly, the device also includes a warning device 40 for outputting a warning W, if it has been determined by checking device 30 that the value of recorded state parameter P is within the specified risk value range. Awakening device 20 is equipped to wake up infant B only after the expiration of a predetermined time period after the output of warning W. The advantage of this specific embodiment is that, because of warning W, first of all caregivers of infant B, such as parents, can be alerted and notified that checking device 30 has determined an indication for a potentially existing SID risk. In this way, compared to the specific embodiment of FIG. 2, the wake-up frequency of infant B may be reduced even further to the absolutely essential measure, and consequently, the sleeping duration of infant B is maximized.

FIG. 4 shows a specific embodiment of the present invention. In this case, the device for preventing sudden infant death also includes an acknowledgment device 50 for acknowledging the taking note of warning W output by the warning device 40. Awakening device 20 is able to be deactivated by acknowledging taking note of warning W within the predetermined time period, so that infant B is not awakened by the acknowledgment of warning W made within the predetermined time period. The output of this warning may take place in such a way that only the caregiver is alerted and awakened, if necessary, without the sleeping infant B being able to perceive the warning. This may be implemented by warning device 40 being located spatially separate from the sleeping infant B (e.g. in the bedroom of the parents), and there drawing attention of the parents or waking them up using an acoustical and/or visual warning signal (e.g. a loud tone and/or a bright light). The warning instruction, in this context, may be transmitted either wire-bound or wireless (e.g. infrared-based or radio wave-based) from checking device 30 or from a central control device, connected to it, to warning device 40. If infant B and parents were to sleep close together (e.g. in one bed), one could use a vibrating warning signal, which does, in fact, vibrate the parents awake, but does, at first, not bother infant B. Because of the time delay of the awakening process, enough time remains for the parents to obtain personally an idea of the state of health of sleeping infant B, and perhaps to deactivate awakening device 20, perhaps before the expiration of the delay time, if the parents determine that everything is in order.

This specific embodiment combines the advantage of the certainty that infant B potentially endangered by SID is awakened in case the parents do not react to the warning, with the advantage of the maximized sleeping duration of infant B.

By contrast to the positioning of warning device 40, in the case of the acknowledgment device 50 it is of advantage if it is located in the vicinity of sleeping infant B. This ensures that the parents must actually make their way to infant B (this is indicated in FIG. 4 by the dashed arrow from the caregiver sleeping, for example, in the parents' bedroom to the position, spatially separated from it, of the caregiver at acknowledgment device 50), in order to break off the automation of the awakening process. Consequently, it is ensured that the parents do not break off the automated awakening process without seeing to the infant. The acknowledgment of the warning signal is able to take place at acknowledgment device 50, via a user interface located there (e.g. a keyboard or a touchscreen).

1-12. (canceled)

13. A device for avoiding sudden infant death, comprising:
a recording device for recording at least one infant-specific state parameter; and
an awakening device for waking up the infant as a function of a value of the recorded state parameter.

14. The device of claim 13, further comprising:
a checking device to check whether the value of the recorded state parameter is inside or outside a predefined value range, which includes all the values of the state parameter that represent a risk for the occurrence of sudden infant death,
wherein the awakening device is configured to wake up the infant if at least this one value of the recorded state parameter lies within this predefined value range.

15. The device of claim 14, further comprising:
a warning device for immediately outputting a warning, if it has been determined by the checking device that the value of the recorded state parameter is within the specified value range,
wherein the awakening device is configured to wake up the infant only after the expiration of a predetermined time period after the output of the warning.

16. The device of claim 15, further comprising:
an acknowledgment device for acknowledging the taking note of the warning output by the warning device,
wherein the awakening device is deactivatable by acknowledging the taking note of the warning within the predetermined time period, so that the infant is not awakened in response to an acknowledgment of the warning that is activated within the predetermined time period.

17. The device of claim 13, wherein the awakening device includes a vibrating mat, which is equipped to wake up the infant using vibrations.

18. The device of claim 13, wherein the awakening device includes at least one of a sound emission and a light emission device, which is configured to wake up the infant using at least one of sound and light.

19. The device of claim 13, wherein the recording device includes at least one sensor unit for measuring at least one of a temperature and a moisture of at least one body part of the infant.

20. The device of claim 13, wherein the recording device includes at least one sensor unit for determining the body position of the infant.

21. The device of claim 20, wherein the sensor unit includes at least one of an acceleration sensor and a gravity sensor for determining the body position of the infant, which is mountable on or in a piece of clothing of the infant or on the body of the infant.

22. The device of claim 20, wherein the sensor unit for determining the body position of the infant includes a heat image camera for taking a heat image of the infant.

23. The device of claim 22, further comprising:
an image processing device for determining the body position of the infant from the heat image data.

24. The device of claim 13, wherein the recording device is connected in a wireless manner or wire-bound at least to the checking device or to a central control device.
